(19) United States
(12) Patent Application Publication
(10) Pub. No.: US 2023/0149333 A1
(43) Pub. Date: May 18, 2023

(54) COMPOSITIONS AND USES OF LOCALLY APPLIED SYNTHETIC AMINO ACID POLYMERS FOR PREVENTION AND TREATMENT OF VIRAL INFECTIONS

(71) Applicant: Amicrobe, Inc., Carlsbad, CA (US)

(72) Inventors: Michael P. Bevilacqua, Boulder, CO (US); Daniel J. Huang, Louisville, CO (US)

(21) Appl. No.: 17/917,858

(22) PCT Filed: Apr. 6, 2021
(86) PCT No.: PCT/US2021/025925

§ 371 Date: Oct. 7, 2022

Related U.S. Application Data
(60) Provisional application No. 63/007,295, filed on Apr. 8, 2020.
(60) Provisional application No. 63/066,294, filed on Aug. 16, 2020.

(51) Int. Cl. A61K 31/198; A61K 9/00; A61K 47/26; A61P 31/14

(52) U.S. Cl. CPC A61K 31/198; A61K 9/007; A61K 9/0034; A61K 9/0043; A61K 47/26; A61P 31/14

(57) ABSTRACT

Antimicrobial pharmaceutical compositions that contain cationic antimicrobials and methods of using them to prevent and/or treat viral infections.

### INCORPORATION BY REFERENCE TO PRIORITY APPLICATIONS

This application claims priority to U.S. Provisional Application Serial No. 63/007295, filed Apr. 8, 2020, and to U.S. Provisional Application Serial No. 63/066294, filed Aug. 16, 2020, both of which are hereby incorporated herein by reference in their entireties.

### BACKGROUND

### Field

This disclosure relates to antimicrobial pharmaceutical compositions that contain cationic antimicrobials and methods of using them to prevent and/or treat viral infections.

### Description

A wide variety of cationic antimicrobials are known for their ability to bind to and disrupt bacterial membranes, including certain antibiotics, bisbiguanides, polymer biguanides, quaternary ammonium compounds, natural antimicrobial peptides, and synthetic cationic polypeptides. A number of publications disclose biological properties of synthetic peptides, including WO 2016/044683, US 2015/0225458 and U.S. Pat. Nos. 7,847,059; 8,088,888; 8,350,003; and 8,470,769.

US Pat. No. 9,017,730 describes synthetic cationic copolypeptides containing varying ratios of cationic amino acid recurring units (such as lysine (K)) and hydrophobic amino acid units (such as leucine (L), isoleucine (1), valine (V), phenylalanine (F) or alanine (A)). US Pat. No. 9,017,730 indicates that poly(L-lysine-HCl)₅₅-block-poly(racemic-hydrophobic amino acid)₂₀, K₅₅(rac-X)₂₀ (for X= A, I, L/F or V), at very low concentration (10 µg/ml), achieved maximum observable (6-log) reduction of bacterial counts for both a Gram-positive (S. aureus) and a Gram-negative (E. coli) bacteria. U.S. Pat. No. 9,017,730 indicates that selected copolypeptides were also shown to be quite effective against other microbes including E. coli O157:H17, as well as other food-borne pathogens, and even against certain endospore forms of microbes. U.S. Pat. No. 9,017,730 indicates that these compounds were also shown to be effective against certain fungal organisms as illustrated for Candida albicans. U.S. Pat. No. 9,017,730 indicates that certain microbial organisms (e.g., P. acnes) may be less sensitive to certain copolypeptides than other microorganisms (e.g., S. aureus). U.S. Pat. No. 9,017,730 indicates that certain solution phase copolypeptides demonstrated antiviral activity against Influenza A virus, with RH/K (partially guanylated lysine) diblock copolypeptide being particularly active.

U.S. Pat. No. 9,446,090 describes synthetic cationic polypeptide(s) along with mutually water-miscible mixtures that contain such a polypeptide and a second pharmaceutically acceptable polymer. Specific examples describe antimicrobial activity against certain bacteria using particular mixtures of synthetic cationic polypeptide(s) with second polymers such as polyethylene glycol (PEG), hydroxyethylcellulose (HEC), and Poloxamer 407.

PCT Publication WO 2018/187617 describes the development of cationic antimicrobial pharmaceutical compositions and methods of use that allow local applications in vivo of doses that provide antimicrobial effectiveness with low risk of local tissue toxicities and/or low risk of systemic / distant organ toxicities. PCT Publication WO 2018/187617 indicates that various embodiments of the cationic antimicrobial pharmaceutical compositions have excellent antimicrobial and safety profiles as demonstrated by successful intraperitoneal application.

While PCT Publication WO 2018/187617 and U.S. Pat. Nos. 9,017,730 and 9,446,090 describe significant advances in the art, a number of challenges remain, particularly with respect to developing pharmaceutically acceptable preparations of locally applied cationic antimicrobials for treating viral infections, such as Coronavirus infections.

### SUMMARY

Antimicrobial pharmaceutical compositions have now been developed that have antiviral activity in addition to their antimicrobial activity. Methods of using such compositions to treat viral infections have now been developed, including treatments for Coronavirus infections. In various embodiments, the antimicrobial pharmaceutical compositions have surprising activity against both Coronavirus and Influenza A virus infections.

An embodiment provides a method of treating a viral infection, comprising:
administering an effective amount of an antimicrobial pharmaceutical composition to a subject in need thereof, wherein:
the antimicrobial pharmaceutical composition comprises:
   an aqueous carrier; and
   an antimicrobial synthetic cationic polypeptide(s) dispersed in the aqueous carrier; and
the antimicrobial synthetic cationic polypeptide(s) comprises a plurality of positively charged amino acid units at neutral pH.

These and other embodiments are described in greater detail below.

### BRIEF DESCRIPTION OF THE DRAWINGS

FIG. 1. Dynamic viscosity, surface tension, and interfacial tension (against n-hexane) of Composition A and Composition B

FIG. 2. Activity of Composition A at various concentrations against S. aureus, S. epidermidis, P. aeruginosa, and C. albicans in a standard 60-minute time-kill assay. Data are presented as log CFU reduction.

FIG. 3. Activity of Composition A at 10 and 100 µg/mL against two S. aureus strains, two MRSA strains, vancomycin-resistant Enterococcus (VRE), and S. pyogenes in a standard 60-minute time-kill assay. Data are presented as log CFU reduction.

FIG. 4. Activity of Composition A at 10 and 100 µg/mL against A. baumannii, pan-resistant A. baumannii, extended spectrum beta-lactamase (ESBL)-positive E. coli, ESBL and Klebsiella pneumoniae carbapenemase-positive K. pneumoniae, P. mirabilis, and S. marcescens in a standard 60-minute time-kill assay. Data are presented as log CFU reduction.

FIG. 5. Activity of Composition A at 10 and 100 µg/mL against P. aeruginosa, multidrug-resistant (MDR) P. aeruginosa, C. koseri, E. aerogenes, S. maltophilia, B. fragilis, and MDR B. fragilis in a standard 60-minute time-kill assay. Data are presented as log CFU reduction.

FIG. 6. Activity of Composition A at 100 µg/mL in water and saline against P. aeruginosa in a standard 60-minute time-kill assay. Data are presented as log CFU survival.

FIG. 7. Activity of Composition B at 100 µg/mL against S. aureus in a standard time-kill assay with 10, 60, and 120-minute exposure times. Data are presented as log CFU survival.

FIG. 8. Activity of Composition B at 100 µg/mL against S. epidermidis in a standard time-kill assay with 10, 60, and 120-minute exposure times. Data are presented as log CFU survival.

FIG. 9. Activity of Composition B at 100 µg/mL against P. aeruginosa in a standard time-kill assay with 10, 60, and 120-minute exposure times. Data are presented as log CFU survival.

FIG. 10. Activity of Composition B at 100 µg/mL against E. coli in a standard time-kill assay with 10, 60, and 120-minute exposure times. Data are presented as log CFU survival.

FIG. 11. Activity of Composition B at 10 and 100 µg/mL against S. aureus in a standard time-kill assay with 10, 60, and 120-minute exposure times. Data are presented as log CFU survival.

FIG. 12. Activity of Composition B at 10 and 100 µg/mL against P. aeruginosa in a standard time-kill assay with 10, 60, and 120-minute exposure times. Data are presented as log CFU survival.

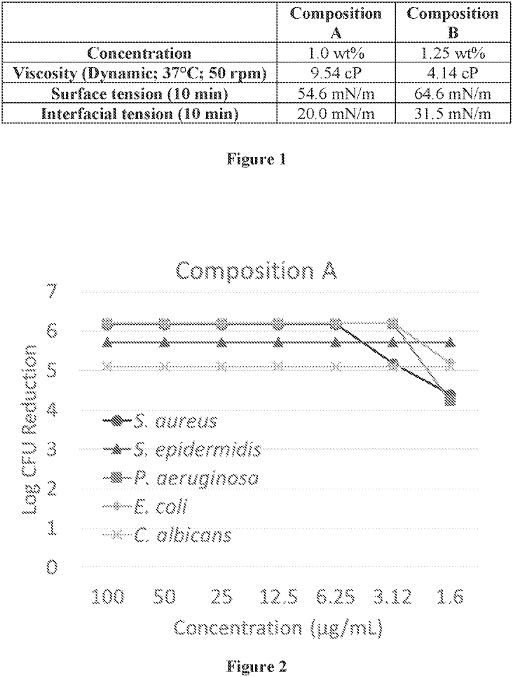

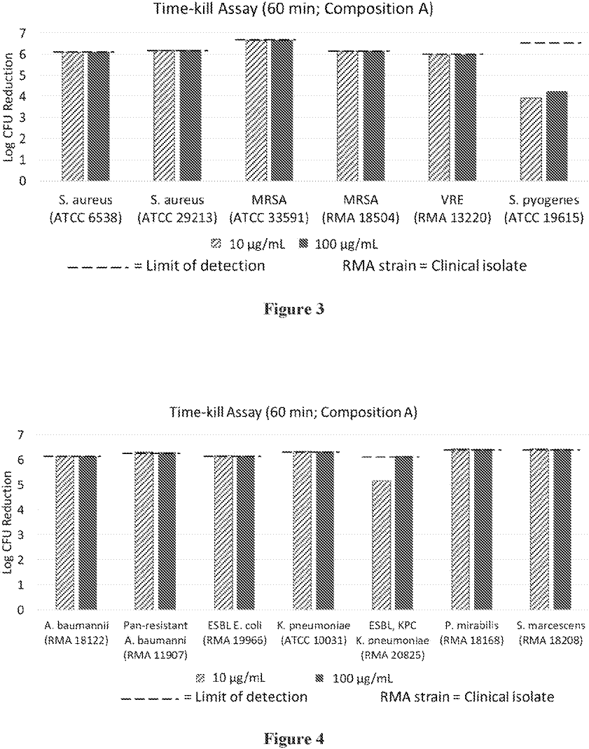

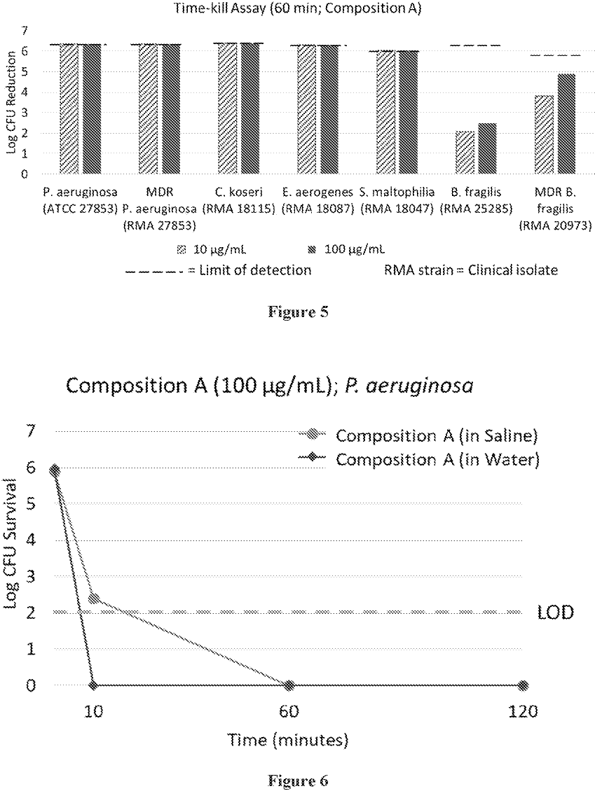

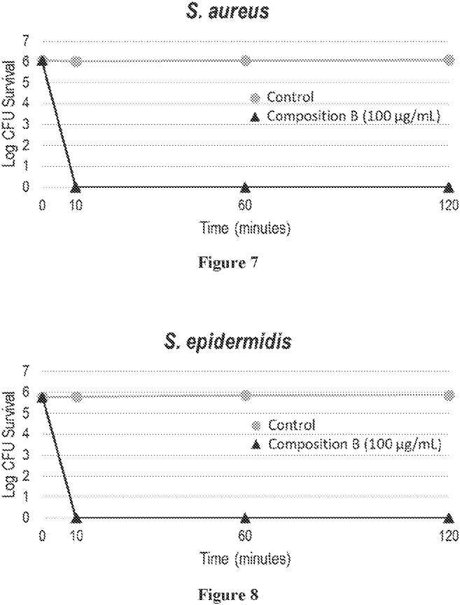

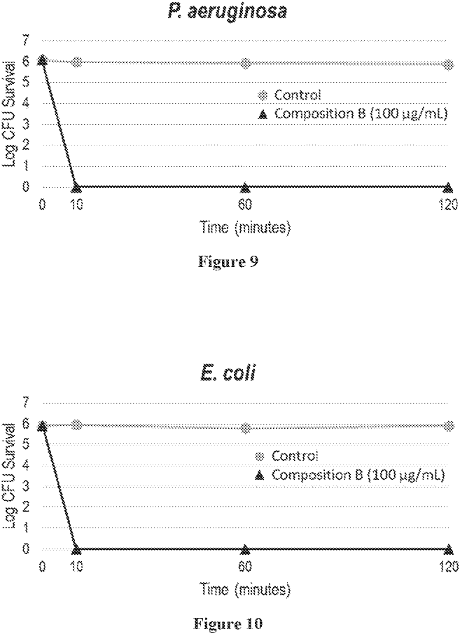

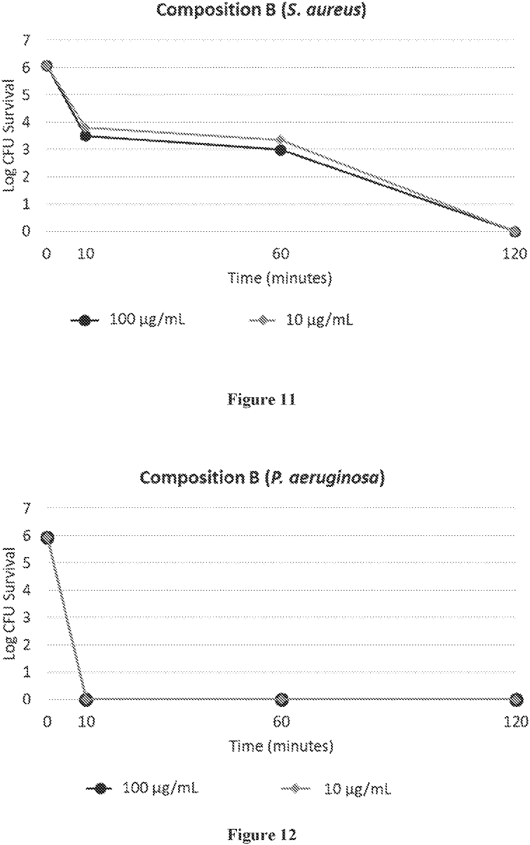

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein in the context of describing antimicrobial synthetic cationic polypeptides, the term “antimicrobial” has its usual meaning as understood by those skilled in the art and thus includes a polypeptide that exhibits microbiocidal activity as determined by a 60 minute time-kill assay against at least one bacteria selected from the group consisting of S. aureus, S. epidermidis, P. aeruginosa, and E. coli.

As used herein in the context of describing antimicrobial synthetic cationic polypeptides, the term “polypeptide” has its usual meaning as understood by those skilled in the art and thus includes a polymer that comprises two or more amino acid recurring units (also referred to as amino acid residues, or more simply units or residues) linked together by peptide bonds. A copolypeptide is a type of polypeptide that comprises two or more different amino acid recurring units. Molecular weights of polymers are weight average as determined by size exclusion chromatography (SEC) with molecular weight standards or using light scattering detection.

The term “block” or “blocky” copolypeptide has its usual meaning as understood by those skilled in the art and thus includes a sequence arrangement of amino acid units that includes a segment (“block”) or segments that is at least 5 amino acid units in length in which the copolypeptide is relatively enriched in one or more of the amino acid units as compared to overall composition of the copolypeptide. In general, synthetic block copolypeptides have a sequence arrangement that reflects deliberate control over the copolymerization process. Likewise, the term “random” copolypeptide has its usual meaning as understood by those skilled in the art and thus includes a sequence arrangement of amino acid units that is a statistical distribution reflecting the concentration of the corresponding amino acid monomers in the polymerization mixture.

As used herein in the context of describing antimicrobial synthetic cationic block copolypeptides, the term “hydrophobic” block has its usual meaning as understood by those skilled in the art and thus includes a sequence arrangement in which a block or segment contains a plurality of hydrophobic amino acid units. Examples of hydrophobic amino acid units are known to those skilled in the art and include glycine (G), leucine (L), isoleucine (I), valine (V), proline (P), tryptophan (W), cysteine (C), methionine (M), phenylalanine (F) and alanine (A). Likewise, the term “hydrophilic” block has its usual meaning as understood by those skilled in the art and thus includes a sequence arrangement in which a block or segment contains a plurality of hydrophilic amino acid units. Examples of hydrophilic amino acid units are known to those skilled in the art and include serine (S), threonine (T), aspartic acid (D) and glutamic acid (E), as well as the positively charged amino acids lysine (K), arginine (R), histidine (H) and ornithine (O).

As used herein in the context of describing antimicrobial synthetic cationic polypeptides, the terms “positively charged” and “cationic” have their usual meanings as understood by those skilled in the art and thus includes an amino acid unit or a polypeptide that is positively charged at neutral pH. Examples of amino acid units that are positively charged at neutral pH include lysine, arginine, histidine and ornithine, and thus the presence of one or more of these positively charged units in the polypeptide (in an amount in excess of any anionic units) can render the polypeptide cationic.

As used herein in the context of describing a sterilized antimicrobial pharmaceutical composition, the term “sterilized” has its usual meaning as understood by those skilled in the art and thus includes a composition that has been subjected to a sterilization process or processes that has the effect of ensuring the absence or reduction of known pathogens in the composition to a degree that renders the sterilized composition clinically acceptable for local administration to a body orifice (such as intranasal administration) or to open skin such as administration to an open wound such as a surgical site. Non-limiting examples of such sterilization processes include heat sterilization (e.g., autoclaving), sterile filtration, irradiation, and/or treatment by chemical agents such as ethylene oxide.

As used herein in the context of describing a self-assembling polypeptide, the term “self-assembling” has its usual meaning as understood by those skilled in the art and thus includes configurations of the polypeptides when dispersed in a medium (such as the other ingredients of a pharmaceutical composition) in which intermolecular attractive forces between certain segments or blocks of the polypeptide causes those segments or blocks to loosely bind to one another. For example, as noted in U.S. Pat. No. 9,017,730, self-assembly of block cationic copolypeptides was observed in aqueous solution, resulting in various hierarchical structures that depended on the configuration of the hydrophobic domains and their effect on attractive intermolecular interactions between the polymer chains. In contrast, U.S. Pat. No. 9,017,730 indicates that random copolypeptides did not exhibit self-assembly. Those skilled in the art are aware of various techniques for determining whether a synthetic cationic polypeptide is self-assembling (see, e.g., U.S. Pat. No. 9,017,730). As compared to an otherwise comparable synthetic cationic polypeptide that exhibits random arrangements in dilute solution and does not exhibit self-assembly, a self-assembling synthetic cationic polypeptide generally exhibits a higher viscosity.

As used herein in the context of describing a molecular feature or parameter that promotes self-assembly of polypeptides, terms such as “promotes” and “promoting” have their usual meaning as understood by those skilled in the art and thus include allowing or enhancing such self-assembly. For example, U.S. Pat. Nos. 9,017,730 and 9,446,090 describe various sequence arrangements of hydrophobic amino acid units and hydrophilic amino acid units that are configured to promote self-assembly of a copolypeptide in water. Similarly, a sterilization technique that is configured to produce a sterilization state that promotes self-assembly of a polypeptide is one that allows for self-assembly or enhances self-assembly when applied to such a polypeptide or to a composition of a polypeptide that is dispersed within an aqueous carrier. Likewise, a composition of an aqueous carrier that is selected to promote self-assembly of a polypeptide is one that allows for self-assembly or enhances self-assembly when such a polypeptide is dispersed within the aqueous carrier.

As used herein in the context of describing a self-assembling synthetic cationic block copolypeptide in comparison to an otherwise comparable random synthetic cationic copolypeptide, the term “otherwise comparable random synthetic cationic copolypeptide” has its usual meaning as understood by those skilled in the art and thus includes copolypeptides that have approximately the same molecular weight and relative numbers of the same hydrophobic and hydrophilic amino acid recurring units as the self-assembling synthetic cationic block copolypeptide, except the sequence arrangement of those amino acid recurring units in the comparable copolypeptide is random rather than block. For example, with respect to a self-assembling block copolypeptide having a hydrophilic (positively charged) lysine block with an average length of about 120 units and a hydrophobic leucine block with an average length of about 30 units, an otherwise comparable random synthetic cationic copolypeptide is one containing an average of about 120 lysine units and about 30 leucine units per copolypeptide chain except that the sequence arrangement of those units along the chain of the random copolypeptide is a statistical distribution reflecting the concentration of the lysine and leucine monomers in the polymerization mixture.

As used herein in the context of describing the administration of a sterilized antimicrobial pharmaceutical composition to a site on a mammalian body in an abundant amount effective to at least partially prevent and/or treat an infection, the terms “abundant” and “abundance” have their usual meaning as understood by those skilled in the art and thus include the administration of amounts of the copolypeptide that are at least 10 times greater than the dosage needed to achieve the desired prevention and/or treatment effect. Typically, a total treatment dose of antimicrobial pharmaceutical composition that includes the administration of 1 g of synthetic cationic polypeptide(s) or more for a 70 kg person, which represents 14.3 mg/kg, is considered to be an abundant administration. Those skilled in the art recognize that biologically active compounds are generally administered in a “therapeutic window” that includes a range of doses over which a desired therapeutic response is achieved without causing significant adverse effects in the subjects to which they are administered. This dosage range is generally between the minimum effective concentration (MEC) and the minimum toxic concentration (MTC) and is typically determined in advance for each biologically active compound, and communicated to the subject and/or caregiver in the form of a dosage recommendation. However, in some situations, such as topical application of an antimicrobial composition to bodily orifices and/or open wounds of mammalian subjects, it may be impractical to determine the MEC and thus highly advantageous to have the flexibility to administer the antimicrobial in abundance. For example, when treating an open wound in an emergency setting where time may be of the essence, it is highly advantageous for a caregiver to have the flexibility to apply the antimicrobial to the open wound in abundance (e.g., in an amount at least ten times greater than the MEC) without being concerned about administering an amount that exceeds the MTC. The MEC for a particular sterilized antimicrobial pharmaceutical composition can be determined by methods known to those skilled in the art, such as those described in the examples below (e.g., amount effective to achieve 3-log CFU killing in an in vitro time-kill assay).

As used herein in the context of describing an antimicrobial pharmaceutical compositions that comprise or consist of an aqueous carrier and an antimicrobial synthetic cationic polypeptide(s) that is dispersed in the aqueous carrier, the term “aqueous carrier” has its usual meaning as understood by those skilled in the art and thus includes various water-based carrier systems that can optionally contain a dispersed substance such as an ionic additive (e.g., a salt) or a non-ionic additive (e.g., polymer, alcohol, sugar and/or surfactant). Substances that are dispersed in the aqueous carrier may be dissolved therein and/or dispersed in the form of small particles.

As used herein in the context of antimicrobial pharmaceutical compositions or other materials having antiviral activity, the term “antiviral” has its usual meaning as understood by those skilled in the art and thus includes an effect of the presence of the antimicrobial pharmaceutical composition or other material that inhibits production of viral particles, typically by damaging viruses directly and/or reducing the number of infectious viral particles formed in a system otherwise suitable for formation of infectious viral particles for at least one virus. In certain embodiments, the antimicrobial pharmaceutical composition is an antiviral composition that has antiviral activity against multiple different viruses (e.g., against both SARS-CoV and SARS-CoV-2).

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage.

### Methods of Treating Viral Infections

Various embodiments provide a method of treating a viral infection, comprising administering an effective amount of an antimicrobial pharmaceutical composition to a subject in need thereof. In various embodiments the viral infection is a Coronavirus infection. As described in greater detail elsewhere herein, in various embodiments the antimicrobial pharmaceutical composition comprises an aqueous carrier and an antimicrobial synthetic cationic polypeptide(s) dispersed in the aqueous carrier. In an embodiment, the antimicrobial synthetic cationic polypeptide(s) comprises a plurality of positively charged amino acid units at neutral pH.

Various embodiments provide a method of treating a viral infection, comprising administering an effective amount of an antimicrobial pharmaceutical composition to a subject in need thereof. In various embodiments the subject is a human. In other embodiments the subject is a non-human primate. The need of the subject for the treatment can be determined in various ways. In various embodiments the subject has tested positive for a viral infection (e.g., a Coronavirus infection), is at risk for a viral infection (e.g., a Coronavirus infection) and/or has symptoms of a viral infection (e.g., a Coronavirus infection). The subject may be in need of treatment for more than one viral infection. For example, in various embodiments, the subject has tested positive for a first viral infection (e.g., a Coronavirus infection), is at risk for a first viral infection (e.g., a Coronavirus infection) and/or has symptoms of a first viral infection (e.g., a Coronavirus infection); and has also tested positive for a second viral infection (e.g., an Influenza A infection), is at risk for a second viral infection (e.g., an Influenza A infection) and/or has symptoms of a second viral infection (e.g., an Influenza A infection).

In an embodiment, the method of treatment comprises identifying the subject on the basis that the subject has, or is at risk of having, one or more risk factors selected from: a positive test for a Coronavirus infection, obesity, diabetes, an advanced age, a cancer, a reduced respiratory function, a reduced cardiovascular function, a reduced kidney function, a nutritional or vitamin deficiency (e.g., vitamin D deficiency), and a reduced immune response function. In an embodiment, the method of treatment comprises identifying a human subject on the basis that the human subject has tested positive for a Coronavirus infection. In an embodiment, the method of treatment comprises administering the antimicrobial pharmaceutical composition to the subject prophylactically. For example, prophylactic treatment may be indicated for a subject having a risk factor listed above. In an embodiment, the prophylactic treatment is administered to coat tissues and physically and/or electrostatically prevent access by viral particles.

In an embodiment, the viral infection is a Coronavirus infection. In an embodiment, the viral infection is an Influenza A virus infection. In another embodiment, the viral infection is not an Influenza A virus infection. In an embodiment, the Coronavirus infection is caused by an α-coronavirus. In another embodiment, the Coronavirus infection is caused by a β-coronavirus. In another embodiment, the Coronavirus infection is caused by a coronavirus selected from CoV 229E, CoV NL63, CoV OC43, CoV HKU1, MERS-CoV, SARS-CoV, and SARS-CoV-2. In another embodiment, the Coronavirus infection is caused by a coronavirus selected from MERS-CoV, SARS-CoV, and SARS-CoV-2. In another embodiment, the Coronavirus infection is caused by MERS-CoV. In another embodiment, the Coronavirus infection is caused by SARS-CoV. In another embodiment, the Coronavirus infection is caused by SARS-CoV-2. In an embodiment, the human subject has the disease COVID-19. In an embodiment, the human subject has the disease SARS. In another embodiment, the human subject has the disease MERS. In another embodiment, the human subject has been identified to have an infection by a coronavirus.

Various routes may be used to administer an antimicrobial pharmaceutical composition to a subject in need thereof. In an embodiment, the antimicrobial pharmaceutical composition is administered to the subject by local application to one or more tissues. For example, in an embodiment, the antimicrobial pharmaceutical composition is administered to the subject by local application to one or more tissues in one or more of an oral cavity, pulmonary cavity, a nasal cavity, a sinus cavity, and/or a vaginal cavity. In an embodiment, the antimicrobial pharmaceutical composition is administered to the subject by a pulmonary route. For example, in an embodiment, the antimicrobial pharmaceutical composition is administered to the subject intranasally and/or by inhalation. In an embodiment, the antimicrobial pharmaceutical composition is administered to an oral cavity of the subject. In another embodiment, the antimicrobial pharmaceutical composition is administered to a vaginal cavity of the subject.

Various embodiments provide a method of treating a Coronavirus infection, comprising administering an effective amount of an antimicrobial pharmaceutical composition to a subject in need thereof, and further comprising administering an effective amount of at least one second Coronavirus treatment to the subject. Various treatment modalities for the second Coronavirus treatment can be used. For example, in an embodiment, the second Coronavirus treatment comprises administering an effective amount of remdesivir to the subject.

### Antimicrobial Pharmaceutical Compositions

Various embodiments provide antimicrobial pharmaceutical compositions that comprise or consist of an aqueous carrier and an antimicrobial synthetic cationic polypeptide(s) dispersed in the aqueous carrier. The amount of cationic polypeptide(s) dispersed in the aqueous carrier can vary over a broad range that depends primarily on the desired viscosity of the antimicrobial pharmaceutical composition. For example, in various embodiments the amount of synthetic cationic polypeptide(s) in the antimicrobial pharmaceutical composition is in the range of about 0.001% to about 10%, by weight based on total weight of the antimicrobial pharmaceutical composition. In some embodiments the amount of synthetic cationic polypeptide(s) dispersed in the aqueous carrier is in the range of about 0.01% to about 5%, by weight based on total weight of the antimicrobial pharmaceutical composition.

In various embodiments the antimicrobial synthetic cationic polypeptide(s) that is dispersed in the aqueous carrier comprises a plurality of positively charged amino acid units (at neutral pH). In an embodiment, the synthetic cationic polypeptide(s) comprises at least 40 amino acid units, of which at least some are positively charged. In an embodiment, the number of positively charged amino acid units in the synthetic cationic polypeptide(s) is at least 5, at least 10, at least 15, or at least 20. Lysine, arginine, histidine and combinations thereof are examples of suitable amino acid units that are positively charged at neutral pH. In an embodiment, the plurality of positively charged amino acid units in the synthetic cationic polypeptide(s) comprises positively charged lysine units.

In various embodiments, the antimicrobial synthetic cationic polypeptide(s) has a viscosity of 2 centistokes (cSt) or greater, as measured at a concentration of 2 wt% in deionized water and at room temperature and/or a temperature of 37° C. Suitable synthetic cationic polypeptide(s) having a range of higher and lower viscosities (e.g., from about 1.5 cSt to about 16,000 cSt, or about 2.0 cSt to about 16,000 cSt) can be made by adjusting the molecular weight of the polypeptide, the level of positively charged amino acid units, and/or the degree to which the polypeptide self-assembles. In an embodiment, the antimicrobial synthetic cationic polypeptide(s) has a viscosity that is greater than that of bovine serum albumin, as measured at a concentration of 2 wt% in deionized water and at room temperature and/or a temperature of 37° C.

In an embodiment, the aqueous carrier, containing the antimicrobial synthetic cationic polypeptide(s) at 2 wt%, has a viscosity at room temperature and/or 37° C. that is greater than that of the aqueous carrier containing albumin at 2 wt% in place of the antimicrobial synthetic cationic polypeptide(s). In an embodiment, the aqueous carrier, containing the antimicrobial synthetic cationic polypeptide(s) at 2 wt%, has a viscosity at room temperature and/or 37° C. that is at least about 20% greater than that of the aqueous carrier containing albumin at 2 wt% in place of the antimicrobial synthetic cationic polypeptide(s). In an embodiment, the aqueous carrier, containing the antimicrobial synthetic cationic polypeptide(s) at 2 wt%, has a viscosity at room temperature and/or 37° C. that is at least about 50% greater than that of the aqueous carrier containing albumin at 2 wt% in place of the antimicrobial synthetic cationic polypeptide(s). In an embodiment, the aqueous carrier, containing the antimicrobial synthetic cationic polypeptide(s) at 2 wt%, has a viscosity at room temperature and/or 37° C. that is at least about 100% greater than that of the aqueous carrier containing albumin at 2 wt% in place of the antimicrobial synthetic cationic polypeptide(s). In an embodiment, the aqueous carrier, containing the antimicrobial synthetic cationic polypeptide(s) at 2 wt%, has a viscosity at room temperature and/or 37° C. that is at least about any one or more of the following values: 3 cSt, 5 cSt, 10 cSt, 25 cSt, 50 cSt, or 100 cSt, or that is within a range defined by endpoints having any two of the aforementioned values.

Antimicrobial synthetic cationic polypeptide(s) can be copolypeptides that comprise other monomer units in addition to the positively charged amino acid units. For example, in various embodiments the antimicrobial synthetic cationic polypeptide(s) may further comprise a plurality of hydrophobic amino acid units. In various embodiments, the number of hydrophobic amino acid units in the cationic copolypeptide is at least 5, at least 10, or at least 15. Examples of suitable hydrophobic amino acid units include leucine (L), isoleucine (l), valine (V), phenylalanine (F), alanine (A), and combinations thereof. In an embodiment, the plurality of hydrophobic amino acid units in the synthetic cationic polypeptide(s) comprises leucine units.

The sequence arrangement of amino acid units in the synthetic cationic polypeptide(s) can be random, blocky or a combination thereof. For example, in an embodiment, the sequence arrangement of hydrophobic amino acid units and positively charged amino acid units in the synthetic cationic polypeptide(s) is blocky. In a number of embodiments, such a block copolypeptide can comprise various hydrophobic and hydrophilic amino acid units. For example, in an embodiment, the synthetic cationic polypeptide(s) is a block copolypeptide that comprises hydrophobic leucine units and positively charged lysine units.

In various embodiments, the antimicrobial synthetic cationic polypeptide(s) self-assembles into multimeric structures in water and other aqueous carriers. Examples of multimeric structures include micelles, sheets, vesicles, and fibrils (see U.S. Pat. No. 9,017,730). In an embodiment, the multimeric structures formed in aqueous media are multimers in solution, micelles, sheets, vesicles, and/or fibrils. In an embodiment, the antimicrobial synthetic cationic polypeptide(s), in deionized water at room temperature and/or 37° C. at a concentration of 3 wt%, forms a self-supporting hydrogel. In an embodiment, the antimicrobial synthetic cationic polypeptide(s) displays surfactant activity in deionized water at room temperature and/or 37° C., as measured by a decrease in surface tension of at least 10% or at least 20% as compared to deionized water alone. In an embodiment, self-assembly of an antimicrobial synthetic cationic polypeptide(s) is evidenced by a critical aggregation concentration for the polypeptide that is below 1000 µg/mL at room temperature and/or 37° C. in deionized water. In an embodiment, self-assembly of an antimicrobial synthetic cationic polypeptide(s) is evidenced by a critical aggregation concentration for the polypeptide that is below 100 µg/mL at room temperature and/or 37° C. in deionized water.

Self-assembly of the antimicrobial synthetic cationic polypeptide(s) can be controlled in various ways. For example, in an embodiment, the antimicrobial synthetic cationic polypeptide(s) comprises a sequence arrangement of hydrophobic amino acid units and positively charged amino acid units that is configured to promote self-assembly of the antimicrobial synthetic cationic polypeptide(s) into multimeric structures. For example, self-assembly of the polypeptide is enhanced by a blocky sequence arrangement of hydrophobic amino acid units and positively charged amino acid units. A higher hydrophobic amino acid unit content and/or longer blocks of hydrophobic amino acid units in the polypeptide tend to enhance self-assembly in aqueous carriers.

The antimicrobial synthetic cationic polypeptide(s) described herein can be dispersed in an aqueous carrier to form antimicrobial pharmaceutical compositions. In various embodiments the aqueous carrier is water. In other embodiments the aqueous carrier is an aqueous solution that comprises a pharmaceutically acceptable salt, a non-ionic additive(s), or a combination thereof. Salt tends to inhibit self-assembly of the polypeptide and thus excessive salt is to be avoided. Normal saline, half normal saline, quarter normal saline and phosphate buffered saline are examples of suitable aqueous carriers that contain a pharmaceutically acceptable salt. In an embodiment, the aqueous carrier comprises sodium chloride.

In various embodiments, the aqueous carrier is an aqueous solution that comprises an additive. Examples of suitable additives include various oils, various other polymers (natural or synthetic), cellulose- or cellulose-derivatives, non-ionic or ionic surfactants, stabilizing agents, viscosity-increasing agents (e.g., polyethylene glycol), various alcohols (including but not limited to stearyl alcohol and/or cetyl alcohol), and combinations thereof. In various embodiments, the aqueous carrier is an aqueous solution that comprises a non-ionic additive. Examples of suitable non-ionic additives include dextrose, mannitol, glycerol, xylitol, sorbitol, surfactant(s), and combinations thereof. Salts and certain sugars or sugar alcohols (e.g., glycerol and xylitol) may be used in amounts effective to modify tonicity and/or osmolality.

The aqueous carrier can comprise various amounts of an additive, such as a pharmaceutically acceptable salt, a non-ionic additive(s), or a combination thereof. In various embodiments, the aqueous carrier comprises an amount of a pharmaceutically acceptable salt that is 9.0 g/L or less, or 8.0 g/L or less; or 7.0 g/L or less, or 6.0 g/L or less; or 5.0 g/L or less, or 4.5 g/L or less; or 4.0 g/L or less, or 3.0 g/L or less. In an embodiment, the amount of additive(s) in the aqueous carrier is selected to control the viscosity of the antimicrobial pharmaceutical composition. In an embodiment, the aqueous carrier comprises an additive in an amount that increases the viscosity of the antimicrobial pharmaceutical composition. In an embodiment, the aqueous carrier comprises an additive in an amount that decreases the viscosity of the antimicrobial pharmaceutical composition. In an embodiment, the non-ionic additive(s) is present in an amount effective to increase the osmotic concentration of the antimicrobial pharmaceutical composition to a value that is at least 10% greater than that of the antimicrobial pharmaceutical composition without said additive(s). In various embodiments the concentration of the additive in the antimicrobial pharmaceutical composition is in the range of about 0.1 wt% to about 10 wt%, based on total weight. In various embodiments the concentration of the non-ionic additive in the antimicrobial pharmaceutical composition is in the range of about 0.01 wt% to about 2 wt%, or in the range of about 0.05 wt% to about 5 wt%, based on total weight.

In various embodiments, an antimicrobial pharmaceutical composition as described herein is sterilized by a sterilization technique(s) configured to achieve a sterilized antimicrobial pharmaceutical composition. In an embodiment, the sterilization technique(s) is configured to have minimal impact on the chemical structure of the synthetic cationic polypeptide and/or the tendency for the synthetic cationic polypeptide to self-assemble. Examples of such sterilization techniques are described in PCT Publication WO 2018/187617. In an embodiment, an antimicrobial pharmaceutical composition as described herein is sterilized by a sterilization technique(s) configured to achieve a sterilized antimicrobial pharmaceutical composition with the antimicrobial synthetic cationic polypeptide(s) having a weight average molecular weight and/or a dispersity comparable to (e.g., within about 10%) that of the antimicrobial synthetic cationic polypeptide(s) of the antimicrobial pharmaceutical composition without sterilization by said sterilization technique(s). In an embodiment, the antimicrobial pharmaceutical composition is sterilized by a sterilization technique(s) configured to achieve a sterilized antimicrobial pharmaceutical composition having a viscosity level at room temperature and/or 37° C. that is comparable to that of the antimicrobial pharmaceutical composition without sterilization by this sterilization technique(s). In an embodiment, the viscosity of the sterilized antimicrobial pharmaceutical composition at room temperature and/or 37° C. is in the range of 20% to 200% of the viscosity of an otherwise comparable unsterilized antimicrobial pharmaceutical composition.

In an embodiment, the antimicrobial pharmaceutical composition has a low toxicity after being infused into the peritoneal cavity of a plurality of mice at a dose of 5 mL/kg, as measured by a mouse survival rate of 50% or greater at 72 hours after being infused. In an embodiment, the antimicrobial pharmaceutical composition has a low toxicity after being infused into the peritoneal cavity of a plurality of mice at a dose of 10 mL/kg, as measured by a mouse survival rate of 50% or greater at 72 hours after being infused. In an embodiment, the antimicrobial pharmaceutical composition has a low toxicity after being infused into the peritoneal cavity of a plurality of mice at a dose of 20 mL/kg, as measured by a mouse survival rate of 50% or greater at 72 hours after being infused. In an embodiment, the antimicrobial pharmaceutical composition has a low toxicity after being infused into the peritoneal cavity of a plurality of mice at a dose of 40 mL/kg, as measured by a mouse survival rate of 50% or greater at 72 hours after being infused. Those skilled in the art appreciate that the dosing may also be expressed in terms of mg/kg instead of mL/kg, and that a mouse survival rate of 50% or greater at 72 hours can include values up to 100%, such as 60% or greater, 70% or greater, 80% or greater, or 90% or greater. For example, in an embodiment, the antimicrobial pharmaceutical composition has a low toxicity after being infused into the peritoneal cavity of a plurality of mice at a dose of 50 mg//kg, as measured by a mouse survival rate of 80% or greater at 72 hours after being infused. In an embodiment, the antimicrobial pharmaceutical composition has a microbiocidal activity that is comparable to that of the otherwise comparable unsterilized antimicrobial pharmaceutical composition, wherein the microbiocidal activity is determined by a 60 minute time-kill assay against at least one bacteria selected from the group consisting of S. aureus, S. epidermidis, P. aeruginosa, and E. coli.

The inclusion of other active pharmaceutical ingredients to the antimicrobial pharmaceutical compositions described herein may enhance antimicrobial performance and / or decrease the risk of toxicities, both local and systemic. In particular, the inclusion of other antimicrobial agents, including antibiotics, antiseptics, iodine compounds, and/or silver compounds, may act cooperatively with the synthetic cationic polypeptide(s) to help prevent and/or treat infection. Further, the inclusion of one or more anti-inflammatory agents may enhance performance and / or decrease the risk of toxicities, both local and systemic. Local inflammation may contribute to pathogenesis of various disease settings that also involve microbial contamination or infection. Examples include otitis externa, chronic sinusitis, pulmonary conditions, and certain wound conditions. Such conditions could be treated by a combination of synthetic cationic polypeptide(s) and anti-inflammatory agents, such as corticosteroids, anti-histamines, and/or anti-cytokines. As such, including anti-inflammatory agents in an antimicrobial composition containing synthetic cationic polypeptide(s) may provide benefits.

Other pharmaceutical ingredients that may be included in the antimicrobial pharmaceutical compositions described herein include lipids, Vitamin D, zinc, sialic acid or sialic acid-containing compounds, nitric oxide or nitric oxide-producing compounds, anesthetics (such as benzocaine), protease inhibitors, mucolytic agents, nucleic acid polymer-disrupting enzymes (such as DNAse), β-agonists (e.g., salmeterol, salbutamol, etc., which may be in amounts effective to raise intracellular cAMP levels), methylxanthines (e.g., theophylline, aminophylline, etc., which may be in amounts effective to inhibit phosphodiesterase and/or increase cAMP levels), PDE4 inhibitors (such as rofulumilast, which may be in amounts effective to increase cilia beat frequency), topical corticosteroids (which may be in amounts effective to provide anti-inflammatory effects and/or increase in cilia beat frequency), cytokines or cytokine inhibitors (including interferons), IL-1 receptor antagonists, IL-1 inhibitors (including anti-IL-1 antibodies), TNF inhibitors (including anti-TNF antibodies), IL-6 inhibitors, and combinations thereof.

In an embodiment, the antimicrobial pharmaceutical composition comprises an anti-inflammatory compound. For example, in an embodiment, the anti-inflammatory compound is selected from the group consisting of a corticosteroid, a histamine inhibitor and a cytokine inhibitor. Examples of corticosteroids include betamethasone dipropionate, clobetasol propionate, diflorasone diacetate, fluocinonide, and halobetasol propionate. Examples of histamine inhibitors include those that inhibit the histamine H1, H2, H3 and H4 receptors. Examples of cytokine inhibitors include glucocorticoids and pentoxifylline.

The antimicrobial pharmaceutical compositions described herein can be made in various ways. In an embodiment, the antimicrobial synthetic cationic polypeptide is made in the general manner taught in PCT Publication WO 2018/187617, U.S. Pat. No. 9,017,730 and/or U.S. Pat. No. 9,446,090, each of which are expressly incorporated herein by reference for all purposes including the teaching of such general methods for making cationic polypeptides and antimicrobial pharmaceutical compositions containing them. The antimicrobial pharmaceutical composition can be made combining the antimicrobial synthetic cationic polypeptide with the aqueous carrier to thereby disperse (e.g., dissolve) the polypeptide in the aqueous carrier. For example, such combining can be accomplished by mixing the ingredients (cationic polypeptide(s), aqueous carrier and optional ingredients such as anti-inflammatory compound) with agitation at a temperature in the range of about 20° C. to 90° C., for a length of time that is effective to disperse (e.g., dissolve) the polypeptide. The ingredients can be mixed together in any order, although those skilled in the art may prefer a particular order in individual cases. Various forms of the antimicrobial pharmaceutical compositions described herein can be made, such as hydrogels, solutions, dispersions, emulsions, dry fibers, dressings, thin films, and/or foams.

### EXAMPLES

Additional embodiments are disclosed in further detail in the following examples, which are not in any way intended to limit the scope of the claims.

### EXAMPLES 1-24

### Antimicrobial Pharmaceutical Compositions

Poly(L-lysine hydrochloride)-b-poly(D,L-leucine) is an antimicrobial synthetic cationic polypeptide that comprises a plurality of positively charged amino acid units at neutral pH. A sample was prepared in accordance with the general procedures described in U.S. Pat. No. 9,017,730. The poly(L-lysine hydrochloride)-b-poly(D,L-leucine) was dispersed in water to form the antimicrobial pharmaceutical composition referred to herein as Composition A. The initial concentration of poly(L-lysine hydrochloride)-b-poly(D,L-leucine) in Composition A was 10 mg/mL. It was subsequently diluted with cell grade water to form solutions having polypeptide concentrations of 1000 µg/mL, 100 µg/mL and 10 µg/mL for evaluation of anti-viral activity.

Poly(L-lysine hydrochloride)-b-poly(L-leucine) is an antimicrobial synthetic cationic polypeptide that comprises a plurality of positively charged amino acid units at neutral pH. A sample was prepared in accordance with the general procedures described in U.S. Pat. No. 9,017,730. The poly(L-lysine hydrochloride)-b-poly(L-leucine) was dispersed in water to form an antimicrobial pharmaceutical composition referred to herein as Composition B. The initial concentration of poly(L-lysine hydrochloride)-b-poly(L-leucine) in Composition B was 10 mg/mL. It was subsequently diluted with cell grade water to form solutions having polypeptide concentrations of 1000 µg/mL, 100 µg/mL and 10 µg/mL for evaluation of anti-viral activity.

### Anti-Viral Activity

The antiviral activities of Composition A and Composition B were evaluated by challenging them with two different viruses, Coronavirus (strain OC43, ZeptoMetrix Corp. #0810024CF) and Influenza A H1N1 (strain A/WS/33, ATCC #VR-1520). A Virucidal Suspension Test (In-Vitro Time-Kill method) based upon the ASTM E1052-20, “Standard Practice to Assess the Activity of Microbicides against Viruses in Suspension” was used. The percent and log₁₀ reductions from the initial populations of the viral strains were determined following exposure to Compositions A and B at two different exposure times (2 minutes and 10 minutes). Tables 1 and 2 summarize the results.

TABLE 1

ACTIVITY AGAINST Coronavirus oc43; Activity of Composition A and Composition B at 10, 100, and 1000 µg/mL. against Coronavirus OC43 in a time-kill assay based upon ASTM E1052-20 with 2- and 10-minute exposures.

| Ex. No. | Composition | Conc. (µg/mL) | Exposure Time (min) | Log₁₀ reduction | Percent reduction |
|---|---|---|---|---|---|
| 1 | Composition A | 1000 | 2 | 0.5 | 68.38 |
| 2 | | | 10 | 1 | 90 |
| 3 | | 100 | 2 | 0.25 | 43.77 |
| 4 | | | 10 | 0.5 | 68.38 |
| 5 | | 10 | 2 | 0.25 | 43.77 |
| 6 | | | 10 | 0.5 | 68.38 |
| 7 | Composition B | 1000 | 2 | 0.5 | 68.38 |
| 8 | | | 10 | 0.75 | 82.22 |
| 9 | | 100 | 2 | 0.75 | 82.22 |
| 10 | | | 10 | 0.75 | 82.22 |
| 11 | | 10 | 2 | 0.25 | 43.77 |
| 12 | | | 10 | 1 | 90 |

TABLE 2

ACTIVITY AGAINST INFLUENZA A H1N1; Activity of Composition A and Composition B at 10, 100, and 1000 ug/mL against Influenza A H1N1 in a time-kill assay based upon ASTM E1052-20 with 2- and 10-minute exposures.

| Ex. No. | Composition | Conc. (µg/mL) | Exposure Time (min) | Log₁₀ reduction | Percent reduction |
|---|---|---|---|---|---|
| 13 | Composition A | 1000 | 2 | 0.75 | 82.22 |
| 14 | | | 10 | 0.50 | 68.38 |
| 15 | | 100 | 2 | 0.75 | 82.22 |
| 16 | | | 10 | 0.75 | 82.22 |
| 17 | | 10 | 2 | 0.25 | 43.77 |
| 18 | | | 10 | 0.50 | 68.38 |
| 19 | Composition B | 1000 | 2 | 0.00 | 0.00 |
| 20 | | | 10 | 1.00 | 90.00 |
| 21 | | 100 | 2 | 0.50 | 68.38 |
| 22 | | | 10 | 0.50 | 68.38 |
| 23 | | 10 | 2 | 0.75 | 82.22 |
| 24 | | | 10 | 0.75 | 82.22 |

The results summarized in Tables 1 and 2 show that antimicrobial pharmaceutical compositions, containing antimicrobial synthetic cationic polypeptides that comprise a plurality of positively charged amino acid units at neutral pH, have surprising antiviral activity over a wide range of polypeptide concentrations against two very different viruses. Coronaviruses constitute the subfamily Orthocoronavirinae in the family Coronaviridae. Coronaviruses are enveloped, positive-sense, single-stranded RNA viruses. Influenza A viruses are species of the genus Alphainfluenzavirus of the virus family Orthomyxoviridae. Influenza A viruses are enveloped, negative-sense, single-stranded, segmented RNA viruses.

What is claimed is:

1. A method of treating a viral infection, comprising:
administering an effective amount of an antimicrobial pharmaceutical composition to a subject in need thereof, wherein:
the antimicrobial pharmaceutical composition comprises:
an aqueous carrier; and
an antimicrobial synthetic cationic polypeptide(s) dispersed in the aqueous carrier;
the antimicrobial synthetic cationic polypeptide(s) comprises a plurality of positively charged amino acid units at neutral pH;
the antimicrobial synthetic cationic polypeptide(s) comprises a plurality of hydrophobic amino acid units; and
the antimicrobial synthetic cationic polypeptide(s) comprises a sequence arrangement of hydrophobic amino acid units and positively charged amino acid units that is configured to promote self-assembly of the antimicrobial synthetic cationic polypeptide(s) into multimeric structures in aqueous media.

2. The method of claim 1, wherein the multimeric structures formed in aqueous media are selected from the group consisting of multimers in solution, micelles, sheets, vesicles, and fibrils.

3. The method of claim 1 or 0, wherein the multimeric structures can be measured by a critical aggregation concentration less than that of a synthetic cationic polypeptide of the same composition with a statistical distribution of amino acids.

4. The method of any one of claims 1 to 3, wherein the antimicrobial synthetic cationic polypeptide(s) self-assembles into multimeric structures, as measured by a critical aggregation concentration that is below 1000 µg/mL at room temperature and/or 37° C. in deionized water.

5. The method of any one of claims 1 to 4, wherein the viral infection is a Coronavirus infection.

6. The method of claim 5, wherein the Coronavirus infection is caused by an α-coronavirus.

7. The method of claim 5, wherein the Coronavirus infection is caused by a β-coronavirus.

8. The method of any one of claims 5 to 7, wherein the Coronavirus infection is caused by a coronavirus selected from CoV 229E, CoV NL63, CoV OC43, CoV HKU1, MERS-CoV, SARS-CoV, and SARS-CoV-2.

9. The method of claim 8, wherein the Coronavirus infection is caused by SARS-CoV-2.

10. The method of any one of claims 1 to 9, wherein the subject is in need of treatment for multiple virus infections.

11. The method of claim 10, wherein one of the multiple virus infections is an influenza A virus infection.

12. The method of any one of claims 1 to 10, wherein the viral infection is not an influenza A virus infection.

13. The method of any one of claims 1 to 12, comprising administering the antimicrobial pharmaceutical composition to the subject by a pulmonary route.

14. The method of claim 13, comprising administering the antimicrobial pharmaceutical composition to the subject intranasally and/or by inhalation.

15. The method of any one of claims 1 to 14, comprising administering the antimicrobial pharmaceutical composition to an oral cavity of the subject.

16. The method of any one of claims 1 to 12, comprising administering the antimicrobial pharmaceutical composition to a vaginal cavity of the subject.

17. The method of any one of claims 1 to 16, comprising administering the antimicrobial pharmaceutical composition to the subject prophylactically.

18. The method of any one of claims 1 to 17, comprising administering the antimicrobial pharmaceutical composition to tissues capable of supporting viral infection and growth to physically and/or electrostatically prevent access by viral particles.

19. The method of any one of claims 5 to 10 or 12 to 18, further comprising administering an effective amount of at least one second Coronavirus treatment to the subject.

20. The method of any one of claims 1 to 19, wherein the subject is a non-human primate.

21. The method of any one of claims 1 to 19, wherein the subject is a human.

22. The method of claim 21, further comprising identifying the human subject on the basis that the subject has, or is at risk of having, one or more risk factors selected from: a positive test for the Coronavirus infection, obesity, diabetes, an advanced age, a cancer, a reduced respiratory function, a reduced cardiovascular function, a reduced kidney function, a nutritional or vitamin deficiency (e.g., vitamin D deficiency), and a reduced immune response function.

23. The method of claim 21 or 22, further comprising identifying the human subject on the basis that the human subject has tested positive for the Coronavirus infection.

24. The method of any one of claims 21 to 23, wherein the human subject has COVID-19.

25. The method of any one of claims 1 to 24, wherein the antimicrobial synthetic cationic polypeptide(s) is dispersed in the aqueous carrier at a concentration in the range of about 0.01% to about 5%, by weight based on total weight of the antimicrobial pharmaceutical composition.

26. The method of any one of claims 1 to 25, wherein the antimicrobial synthetic cationic polypeptide(s) at a concentration of 2 wt% in deionized water has a viscosity at room temperature and/or 37° C. of 2 centistokes (cSt) or greater.

27. The method of any one of claims 1 to 26, wherein the aqueous carrier, containing the antimicrobial synthetic cationic polypeptide(s) at 2 wt%, has a viscosity at room temperature and/or 37° C. that is greater than that of the aqueous carrier containing albumin at 2 wt% in place of the antimicrobial synthetic cationic polypeptide(s).

28. The method of any one of claims 1 to 27, wherein the aqueous carrier, containing the antimicrobial synthetic cationic polypeptide(s) at 2 wt%, has a viscosity at room temperature and/or 37° C. that is at least about 5 cSt.

29. The method of any one of claims 1 to 28, wherein the aqueous carrier, containing the antimicrobial synthetic cationic polypeptide(s) at 2 wt%, has a viscosity at room temperature and/or 37° C. that is at least about 10 cSt.

30. The method of any one of claims 1 to 29, wherein the aqueous carrier, containing the antimicrobial synthetic cationic polypeptide(s) at 2 wt%, has a viscosity at room temperature and/or 37° C. that is at least about 25 cSt.

31. The method of any one of claims 1 to 30, wherein the aqueous carrier, containing the antimicrobial synthetic cationic polypeptide(s) at 2 wt%, has a viscosity at room temperature and/or 37° C. that is at least about 50 cSt.

32. The method of any one of claims 1 to 31, wherein the antimicrobial pharmaceutical composition has a low toxicity after being infused into the peritoneal cavity of a plurality of healthy, young adult mice at a dose of 50 mg/kg, as measured by a mouse survival rate of 80% or greater at 72 hours.

33. The method of any one of claims 1 to 32, wherein the plurality of positively charged amino acid units at neutral pH is at least 5.

34. The method of any one of claims 1 to 33, wherein the aqueous carrier is water or an aqueous solution of a pharmaceutically acceptable salt.

35. The method of any one of claims 1 to 34, wherein the aqueous carrier further comprises a non-ionic additive(s).

36. The method of claim 35, wherein the non-ionic additive(s) is present in an amount effective to increase the osmotic concentration of the antimicrobial pharmaceutical composition to a value that is at least 10% greater than that of the antimicrobial pharmaceutical composition without said additive(s).

37. The method of claim 35 or 36, wherein the non-ionic additive is selected from the group consisting of dextrose, mannitol, glycerol, xylitol, sorbitol, surfactant(s), and combinations thereof.

38. The method of any one of claims 1 to 37, wherein the aqueous carrier further comprises an additive in an amount that increases the viscosity of the antimicrobial pharmaceutical composition.

39. The method of any one of claims 1 to 37, wherein the aqueous carrier further comprises an additive in an amount that decreases the viscosity of the antimicrobial pharmaceutical composition.

40. The method of any one of claims 1 to 39, wherein the antimicrobial pharmaceutical composition is sterilized by a sterilization technique(s) configured to achieve a sterilized antimicrobial pharmaceutical composition with the antimicrobial synthetic cationic polypeptide(s) having a weight average molecular weight comparable to that of the antimicrobial synthetic cationic polypeptide(s) of the antimicrobial pharmaceutical composition without sterilization by said sterilization technique(s).

41. The method of any one of claims 1 to 40, wherein the antimicrobial pharmaceutical composition is sterilized by a sterilization technique(s) configured to achieve a sterilized antimicrobial pharmaceutical composition with the antimicrobial synthetic cationic polypeptide(s) having a weight average molecular weight and dispersity comparable to that of the antimicrobial synthetic cationic polypeptide(s) of the antimicrobial pharmaceutical composition without sterilization by said sterilization technique(s).

42. The method of any one of claims 1 to 41, wherein the antimicrobial pharmaceutical composition is sterilized by a sterilization technique(s) configured to achieve a sterilized antimicrobial pharmaceutical composition having a viscosity level at room temperature and/or 37° C. that is comparable to that of the antimicrobial pharmaceutical composition without sterilization by said sterilization technique(s).

43. The method of any one of claims 40 to 42, wherein the viscosity of the sterilized antimicrobial pharmaceutical composition at room temperature and/or 37° C. is in the range of 20% to 200% of the viscosity of an otherwise comparable unsterilized antimicrobial pharmaceutical composition.

44. The method of any one of claims 1 to 43, wherein the antimicrobial pharmaceutical composition has a microbiocidal activity that is comparable to that of the otherwise comparable unsterilized antimicrobial pharmaceutical composition, wherein the microbiocidal activity is determined by a 60 minute time-kill assay against at least one bacteria selected from the group consisting of S. aureus, S. epidermidis, P. aeruginosa, and E. coli.

45. The method of any one of claims 1 to 44, wherein the plurality of hydrophobic amino acid units comprises at least 5 hydrophobic amino acid units selected from leucine (L), isoleucine (1), valine (V), phenylalanine (F) or alanine (A).

46. The method of claim 45, wherein the plurality of hydrophobic amino acid units comprises at least 10 hydrophobic amino acid units selected from leucine (L), isoleucine (1), valine (V), phenylalanine (F) or alanine (A).

47. The method of any one of claims 45 to 46, wherein the plurality of hydrophobic amino acid units comprises at least 15 hydrophobic amino acid units selected from leucine (L), isoleucine (1), valine (V), phenylalanine (F) or alanine (A).

48. The method of any one of claims 45 to 47, wherein the hydrophobic amino acid units comprise leucine units.

49. The method of any one of claims 1 to 48, wherein the plurality of positively charged amino acid units comprises lysine units.

50. The method of any one of claims 1 to 49, wherein the antimicrobial synthetic cationic polypeptide(s) is a block copolypeptide that comprises hydrophobic leucine units and positively charged lysine units.

51. The method of any one of claims 1 to 50, wherein the antimicrobial synthetic cationic polypeptide(s) comprises at least 40 amino acid units.

52. The method of any one of claims 1 to 51, wherein the antimicrobial synthetic cationic polypeptide(s), in deionized water at room temperature and/or 37° C. at a concentration of 3 wt%, forms a self-supporting hydrogel.

53. The method of any one of claims 1 to 51, wherein the antimicrobial synthetic cationic polypeptide(s) displays surfactant activity in deionized water at room temperature and/or 37° C. at a concentration of 1 wt%, as measured by a decrease in surface tension of at least 10% as compared to deionized water alone.

54. The method of any one of claims 1 to 53, wherein the antimicrobial pharmaceutical composition further comprises an anti-inflammatory compound.

55. The method of claim 54, wherein the anti-inflammatory compound is selected from the group consisting of a corticosteroid, a histamine inhibitor and a cytokine inhibitor.
